(19) United States
(12) Patent Application Publication
(10) Pub. No.: US 2005/0010214 A1
(43) Pub. Date: Jan. 13, 2005

(54) System of holding at least two vertebrae together for the purpose of spinal osteosynthesis

(76) Inventor: Jean-Louis Tassin, Metz (FR)

Correspondence Address: BACHMAN & LAPOINTE, P.C., 900 CHAPEL STREET, SUITE 1201, NEW HAVEN, CT, 06510, US

(21) Appl. No.: 10/492,772

(22) PCT Filed: Oct. 17, 2002
(86) PCT No.: PCT/FR02/03564

(30) Foreign Application Priority Data
Oct. 17, 2001 (FR) 01/13388

(51) Int. Cl. A61B 17/56; A61F 2/30

(52) U.S. Cl. 606/61

(57) ABSTRACT

The invention relates to a spinal osteosynthesis system which is intended for the lumbar region in particular. The inventive system is of the type that comprises: at least one connecting rod consisting of one main part having an axis; at least one pedicular screw comprising a bone threading which penetrates a vertebra; and at least one connector which is used respectively to connect a pedicular screw to the connecting rod, each connector comprising first fixing means for fixing the connector to the connecting rod and second fixing means for fixing the connector to a pedicular screw. The invention is characterized in that the aforementioned connecting rod is provided with at least one secondary part having an axis and at least one turning point such that the axis is oriented at a non-zero angle α in relation to an axis. Moreover, at least one end connector is provided at the free end of the secondary part forming one piece therewith.

### BACKGROUND OF THE INVENTION

The present invention concerns systems used for holding at least two vertebrae of a vertebral column together for the purpose of spinal osteosynthesis, for example in a human, the aim being to eliminate, for example, the cause of the pain generated by a damaged intervertebral disk or resulting from an injury.

Practitioners in the field of bone surgery use, in particular, spinal osteosynthesis systems comprising at least one metal rod which is fixed on the vertebrae with the aid of pedicle screws or similar, via suitable connectors which securely connect the rod and the screws.

To perform such osteosynthesis, the practitioner generally secures two rods to the two vertebrae, these two rods being situated on each side of the spinous processes.

The prior art in this regard includes European patent application EP 301 489, French patent application FR 2 687 561 and American patent U.S. Pat. No. 5,947,965.

The systems which have been used hitherto give good results but can have some disadvantages, particularly with regard to fitting them in place, because the sites for fixing the pedicle screws are generally difficult to reach and the processes of the vertebrae make passage of the metal rods difficult.

Moreover, the ends of these rods, and the connectors, sometimes abut against, or at least rub against, the articular facets of the vertebra which is located above the treated zone and is not the subject of osteosynthesis, and they can thus impede their movement and cause their degeneration.

This is more especially the case when osteosynthesis is performed in the lumbar region, and this is obviously a drawback which can, in the short term, be very damaging for the patient.

### SUMMARY OF THE INVENTION

It is therefore an object of the present invention to produce a system for holding at least two vertebrae together, which system overcomes the disadvantages of the similar systems of the prior art mentioned above.

More precisely, the object of the present invention is to preserve the articulation.

The present invention relates in its widest sense to a spinal osteosynthesis system, intended for the lumbar region in particular, and of the type comprising:
at least one connecting rod having a main part with an axis,
at least one pedicle screw comprising a bone thread for penetrating into a vertebra,
at least one connector for respectively connecting a pedicle screw to said connecting rod, each connector comprising first fixing means for fixing said connector to the connecting rod, and second fixing means for fixing said connector to a pedicle screw,
characterized in that said connecting rod has at least one secondary part with an axis and at least one bend point such that the axis is oriented at a non-zero angle α in relation to the axis, the free end of said secondary part being formed in one piece with an end connector.

The end connector is preferably positioned at one end of the secondary part of the connecting rod in such a way that the axis intersects the axis of the pedicle screw of said end connector.

The second fixing means for joining the pedicle screw to the end connector preferably consist of:
a swivel joint forming a male part intended to cooperate with a female part formed in said connector, and
locking means with which it is possible to exert a pressure on said male part when it is positioned in said female part.

The male part of the swivel joint preferably consists of a hollow bulb, the internal cross section of said bulb substantially complementing the external cross section of the pedicle screw, the maximum external cross section S of said bulb being slightly smaller than the internal cross section of said female part of said connector in order to permit pivoting of said bulb inside said female part on substantially the axis of said female part.

The female part of the swivel joint preferably consists of a through-orifice formed in the body of the connector, the internal cross section of this through-orifice being at least equal to the maximum external cross section S of the deformable bulb.

The locking means with which it is possible to exert a pressure on said male part when it is positioned in said female part preferably consist of:
a threaded hole with an axis inclined at a non-zero angle β in relation to the axis of the female part, and
a locking screw which is screwed into the threaded hole,
said threaded hole opening into said female part in such a way as to allow one end of said locking screw to exert a pressure against said male part when the locking screw is screwed into the threaded hole.

The outer wall of the bulb preferably comprises at least one weakening groove.

The angle β between the axis of the threaded hole and the axis of the female part is preferably substantially 30 degrees.

The axis of the female part of the swivel joint is preferably substantially perpendicular to the plane defined by the axis of the main part of the connecting rod and the axis of the secondary part.

The angle α between the axis of the female part of the swivel joint and the axis of the main part of the connecting rod is preferably substantially 30 degrees.

The cross section of the secondary part preferably increases toward the end.

The present invention thus advantageously permits locking of compression/distraction, while leaving the articular facets free.

### BRIEF DESCRIPTION OF THE DRAWINGS

Other characteristics and advantages of the invention will become clear from the following description which is given by way of illustration and is non-limiting and in which reference is made to the attached drawings, in which:

FIG. 1 shows the operating principle of an embodiment of part of the system according to the invention for holding at least two vertebrae together, the system being shown implanted on a vertebra which is to be the subject of osteosynthesis and which is bordered by another vertebra, not the subject of this osteosynthesis;

FIG. 2 shows a plan view of a possible industrial embodiment of part of the system according to the invention;

FIG. 3 shows an exploded view of the part of the system according to the embodiment illustrated in FIG. 2; and

FIG. 4 shows a cross section through this same part in the same embodiment, said cross section being labeled IV-IV in FIG. 2.

### DETAILED DESCRIPTION OF THE PREFERRED EMBODIMENT(S)

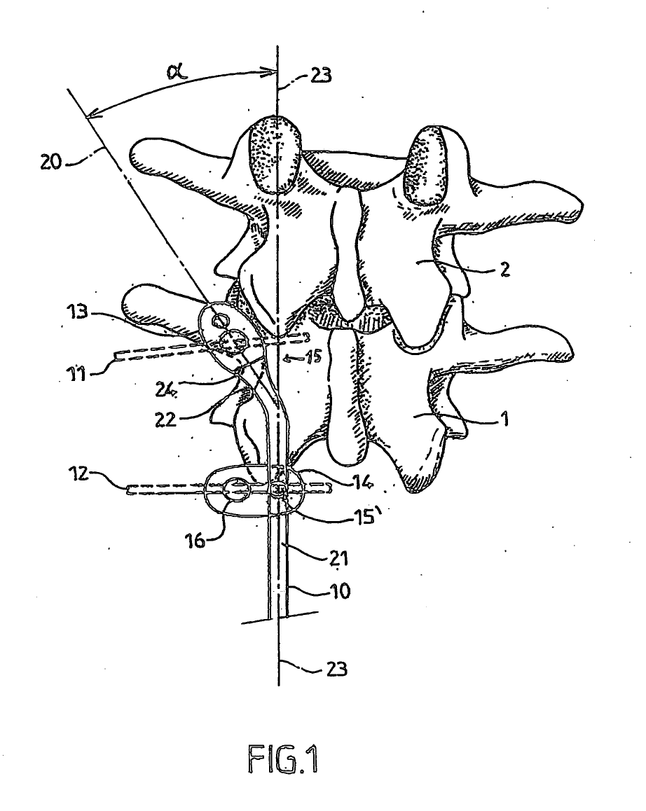

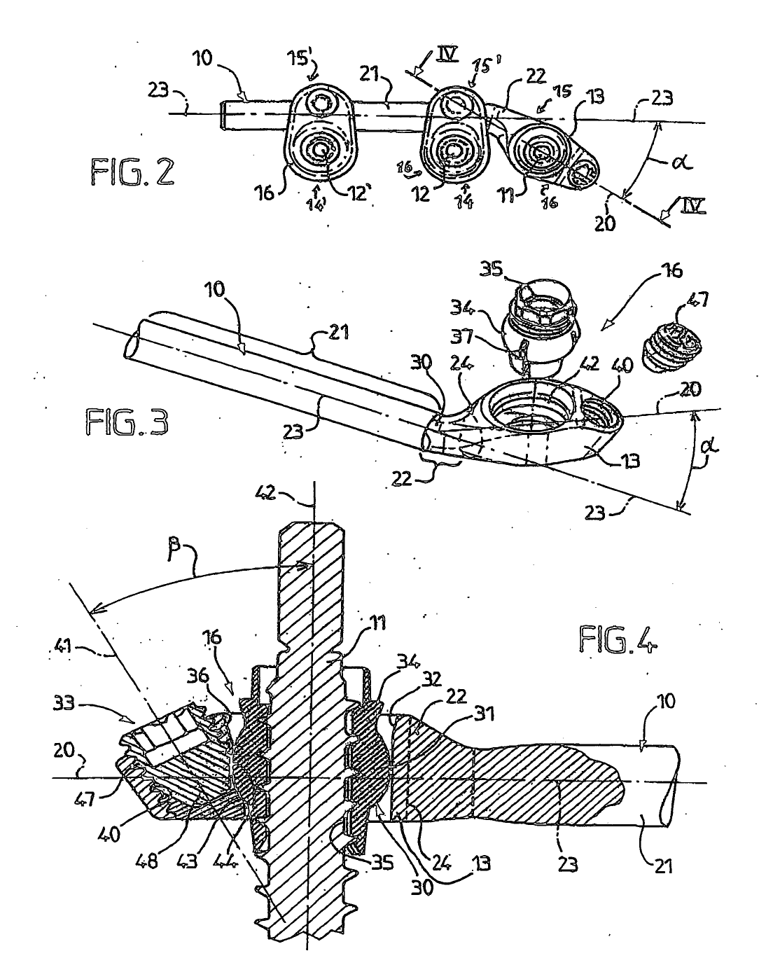

FIG. 1 shows the operating principle of an embodiment of the system according to the invention for holding at least two vertebrae together, and FIGS. 2 to 4 show a possible industrial embodiment of this system. However, to make it easier to understand the present description, the same reference labels designate the same elements irrespective of the figure in which they appear and irrespective of the form in which these elements are shown.

This having been made clear, the present invention concerns a system of holding at least two vertebrae together for obtaining spinal osteosynthesis, FIG. 1 showing only one, labeled (1), of these two vertebrae, the vertebra labeled (2) being a vertebra which is not the subject of osteosynthesis and which lies adjacent to the vertebra (1).

Referring more particularly to FIG. 1, the system comprises at least one connecting rod or bar (10), for example of titanium or similar, at least one pedicle screw (11, 12, 12′), two screws (11, 12) being illustrated diagrammatically in this figure, each screw comprising a shank with a bone thread and, if appropriate, a maneuvering head.

The system additionally comprises at least one connector (13, 14, 14′), two connectors (13, 14) being shown diagrammatically in FIG. 1, and three connectors (13, 14, 14′) being shown diagrammatically in FIG. 2, for respectively connecting the pedicle screws (11, 12, 12′) to said connecting rod (10).

Each connector (13, 14, 14′) comprises first fixing means (15, 15′) for fixing said connector (13, 14, 14′) to the connecting rod (10), and second fixing means (16) for fixing said connector (13, 14, 14′, etc.) to a pedicle screw (11, 12, 12′).

According to the invention, said connecting rod (10) comprises a main part (21) with an axis (23). Said connecting rod (10) also comprises at least one secondary part (22) with an axis (20) and at least one bend point (30) in such a way that the axis (20) is oriented at a non-zero angle α in relation to the axis (23), the free end of said secondary part (22) moreover being formed in one piece with at least one end connector (13), that is to say at the end away from the main part (21).

As is illustrated in all the figures, the system thus comprises an end connector (13) which is joined non-removably to the secondary end part (22) of the connecting rod (10).

This end connector (13) is positioned at one end (24) of the secondary part (22) of the connecting rod (10) in such a way that the axis (20) of the secondary part (22) at one point intersects the axis of the pedicle screw (11) of said end connector (13).

The first fixing means (15) defined above, for joining the connector (13) to the secondary end part (22) of the rod (10), then consist of the secondary part (22) of the rod and the connector (13) being made in one piece, as is illustrated in FIGS. 1 to 4, for example by machining or more generally by a forging process or similar. It is possible for the cross section of the secondary part (22) to increase toward the end (24).

The connectors (14, 14′) preferably comprise first fixing means (15′) and second fixing means (16) of the type known from international patent application No. WO 01/3977 and in particular from the version illustrated in FIGS. 1 to 9, incorporated herein by way of reference.

In a preferred embodiment, the second fixing means (16) defined above, for joining the pedicle screws (11) to the connectors (13), consist of:
a swivel joint (30) forming a male part (31) intended to cooperate with a female part (32) formed in said connector (13), and
locking means (33) with which it is possible to exert a pressure on said male part (31) when it is positioned in said female part (32).

The male part (31) of the swivel joint (30) preferably consists of a hollow and deformable bulb (34) which has a substantially tubular overall shape with end cross sections which are smaller than the cross section of the central part, and with a through-bore (35). The internal cross section of said bulb (34) substantially complements the external cross section of the pedicle screw (11), the maximum external cross section S of said bulb (34) being slightly smaller than the internal cross section of said female part (32) of said connector (13) in order to allow pivoting of said bulb (34) inside said female part (32) on substantially the axis (42) of said female part (32).

The female part (32) of the swivel joint (30) of the connector (13) is formed by a through-orifice (36) produced directly in the body of the connector (13), the cross section of this through-orifice being at least equal to the maximum external cross section S of the deformable bulb (34), before deformation, so that, as will be explained hereinafter in the description of a method of use of the system according to the invention, the deformable bulb is able, at least initially, to pivot in the through-orifice (36).

In a preferred embodiment, the axis (42) of the female part (32) is substantially perpendicular to the plane defined by the axis (23) of the main part (21) of the connecting rod (10) and the axis (20) of the secondary part (22), as is illustrated in FIGS. 2 to 4.

In the case of the preferred embodiment of the swivel joint (30) described above, the means (33) for locking the following four elements—the male part (31) and the female part (32) of the swivel joint (30), the pedicle screw (11) and the connector (13)—advantageously consist of:
a threaded hole (40) with an axis (41) inclined by a non-zero angle β in relation to the axis (42) of the female part (32), and
a locking screw (47) which is screwed into the threaded hole (40),
said threaded hole (40) opening into said female part (32) in such a way as to allow one end (48) of said locking screw (47) to exert a pressure against said male part (31) when the locking screw (47) is screwed into the threaded hole (40).

The threaded hole (40) and the through-orifice (36) thus have a common wall part or an intersection aperture (44). The connector (13) thus comprises a stop (43) for determining the position of the bulb (34) in the through-orifice (36) in such a way that part of this bulb is situated on the common wall part (44).

In a preferred embodiment, the stop (43) for determining the position of the bulb (34) in the through-orifice (36) so that part of this bulb is situated on the common wall part (44) is formed by a portion of the body of the connector projecting into the through-orifice (36), this portion having a substantially spherical shape so that the bulb (34) can pivot in the orifice (36) while at the same time being maintained in position there, opposite the threaded hole (40).

To make it easier to fit the pedicle screw (11) in the vertebra (1) in cooperation with the swivel joint (30) and the connector (13), it is very advantageous, as is illustrated in FIG. 4, that the through-bore (35) produced in the deformable bulb (34) comprises a thread substantially complementing that of the shank of the pedicle screw (11), in order to easily displace the pedicle screw with respect to the connector.

The deformable bulb can be produced in different ways, but it will advantageously be made of a biocompatible material such as titanium or similar, and, in order to promote its at least partial deformation, on account of the hardness of the titanium, its external lateral wall will then comprise at least one weakening groove (37), and preferably four weakening grooves (37) which are arranged at uniform angles in relation to the axis of the through-bore (35), as is illustrated in FIG. 3.

The Applicant has achieved good results when the non-zero angle β between the axis (41) of the threaded hole (40) and the axis (42) of the through-orifice (36) is substantially equal to thirty degrees, the non-zero angle α between the axis (20) of the secondary part (22) of the connecting rod (10) and the axis (23) of the main part (21) of said rod also being substantially equal to thirty degrees.

In the embodiment illustrated in the figures, the secondary part (22) of the connecting rod (10) has been shown as being rectilinear. However, it will be obvious that, if necessary, it could have a curvature, its axis (20) defined above then representing its mean direction in relation to the main part (21) of the connecting rod.

The system according to the invention, as it has been described above, is used and functions in the following way:

When a practitioner wishes to perform osteosynthesis between at least two vertebrae (1) using a system according to the invention in its industrial embodiment, as is illustrated more particularly in FIGS. 2 to 4, he positions the connecting rod (10) in the manner indicated above and fixes the main part (21) of this connecting rod to the vertebrae (1) to be joined by osteosynthesis, by means of several connectors (14, 14′) and pedicle screws (12, 12′) in the same way as in the systems in the prior art.

However, because of the structure described above, when the connecting rod (10) is positioned in this way, the curved secondary part (22), with its connector (13), is situated away from the articular facet of the superior vertebra (2) which immediately follows the treatment zone, thus not interfering with its displacement.

The practitioner then places the pedicle screw (11) in line with the through-orifice (36), with the bulb (34) already screwed for example on the start of the thread of the shank.

He engages this assembly in the through-orifice (36) until the bulb abuts against the stop (43) in the form of a spherical wall portion, the penetrating end of the pedicle screw (11) initially not making contact with the osseous part of the vertebra (1) in which it is to be implanted, because of the initial position of the bulb on the threaded shank of the screw.

Then, by maneuvering its head, the practitioner gives the pedicle screw (11) the correct angular position chosen for its implantation in the osseous part of the vertebra (1), the pivoting of the pedicle screw being facilitated by the spherical shape of the bulb mounted so as to cooperate with it in the through-orifice (36), constituting a swivel joint as defined above.

When the practitioner has found the correct angular position of the pedicle screw (11) in relation to the osseous part of the vertebra (1), he turns the screw so as to cause it to penetrate with self-tapping into the vertebra, the threaded shank of this pedicle screw (11) being able to move in translation relative to the bulb (34) since the through-bore (35) of the bulb comprises a complementary thread and the bulb is retained by the stop (43), as has been explained above.

When the pedicle screw (11) has sufficiently penetrated into the vertebra (1), the practitioner screws the locking screw (47) in until its end (48) abuts with force against the part of the bulb (34) passing through the common wall part (44). Tightening this locking screw with a given force sufficiently deforms the bulb (34) in order to, as it were, crimp it on the threaded shank of the pedicle screw (11), and likewise acts on the end (48) of the locking screw (47) in relation to the body of the bulb, thereby providing a rigid assembly of pedicle screw (11), bulb (34), connector (13) and locking screw (47), as is required to achieve osteosynthesis.

The surgical technique described above is not limiting; it is given only by way of example. In particular, the pedicle screws are fitted in cooperation with the swivel joint (30) and thereafter screwed into the vertebrae. This possibility is offered by the absence of a head on the pedicle screw. A technique in which the pedicle screws are fitted first in the vertebrae, and the swivel joints are then fitted on the pedicle screws, is also entirely possible.

Of course, the parameters of the implanted system, namely the length of the curved secondary part (22) of the connecting rod (10) and the exact value of the angle α of the system will be chosen by the practitioner in accordance with the physiology of the vertebrae (1, 2, etc.) of the vertebral column to be treated, and advantageously prior to fitting the osteosynthesis system, so as not to prolong the time of the operation, and so as to avoid excessive trauma for the patient. To do this, a range of systems with different values of these parameters is made available to the practitioner.

1-11. (cancelled)

12. A spinal osteosynthesis system comprising:
at least one connecting rod having a main part with an axis,
at least one pedicle screw comprising a bone thread for penetrating into a vertebra,
at least one connector for respectively connecting said at least one pedicle screw to said at least one connecting rod, each said connector comprising a first means for fixing said connector to the at least one connecting rod, and second means for fixing said connector to said at least one pedicle screw, and
each said connecting rod having at least one secondary part with an axis and at least one bend point such that the secondary part axis is oriented at a non-zero angle α in relation to the axis of the at least one connecting rod, the free end of said at least one secondary part being formed in one piece with at least one end connector.

13. The spinal osteosynthesis system as claimed in 12, wherein each said end connector is positioned at one end of the at least one secondary part in such a way that the secondary part axis intersects an axis of the at least one pedicle screw of said at least one end connector.

14. The spinal osteosynthesis system as claimed in claim 12, wherein said second fixing means consists of a swivel joint forming a male part intended to cooperate with a female part formed in said at least one connector, and locking means for exerting a pressure on said male part when positioned in said female part.

15. The spinal osteosynthesis system as claimed in claim 14, wherein the male part consists of a hollow bulb having an internal cross section which substantially complements an external cross section of the at least one pedicle screw and a maximum external cross section S of said bulb being slightly smaller than an internal cross section of said female part in order to permit pivoting of said bulb inside said female part on substantially an axis of said female part.

16. The spinal osteosynthesis system as claimed in claim 15, wherein the female part consists of a through-orifice formed in a body of the at least one end connector, and an internal cross section of said through-orifice being at least equal to the maximum external cross section S of the bulb.

17. The spinal osteosynthesis system as claimed in claim 14, wherein the locking means consists of a threaded hole with an axis inclined at a non-zero angle β in relation to an axis of the female part, and a locking screw which is screwed into the threaded hole, said threaded hole opening into said female part in such a way as to allow one end of said locking screw to exert a pressure against said male part when the locking screw is screwed into the threaded hole.

18. The spinal osteosynthesis system as claimed in claim 15, wherein an outer wall of the bulb has at least one weakening groove.

19. The spinal osteosynthesis system as claimed in claim 17, wherein the angle β between the axis of the threaded hole and the axis of the female part is substantially 30 degrees.

20. The spinal osteosynthesis system as claimed in claim 14, wherein an axis of the female part is substantially perpendicular to a plane defined by an axis of the main part of the at least one connecting rod and the axis of the secondary part.

21. The spinal osteosynthesis system as claimed in claim 12, wherein the angle α is substantially 30 degrees.

22. The spinal osteosynthesis system as claimed in claim 12, wherein a cross section of the secondary part increases toward an end.
